(19) United States
(12) Plant Patent Application Publication
(10) Pub. No.: US 2018/0263166 P1
(43) Pub. Date: Sep. 13, 2018

(54) Verbena plant named 'KLEVP16485'

(50) ;

(71) Applicant: Klemm+Sohn GmbH & Co. KG, Stuttgart (DE)

(72) Inventor: Ruijun Li, North Parramatta (AU)

(73) Assignee: Klemm+Sohn GmbH & Co. KG, Stuttgart (DE)

(21) Appl. No.: 15/530,847

(22) Filed: Mar. 7, 2017

(51) Int. Cl. A01H 6/86

(52) U.S. Cl. PLT/308

(57) ABSTRACT

A new verbena plant particularly distinguished by dark violet flowers with white eyes, a semi-upright growth habit, and good tolerance to powdery mildew, is disclosed.

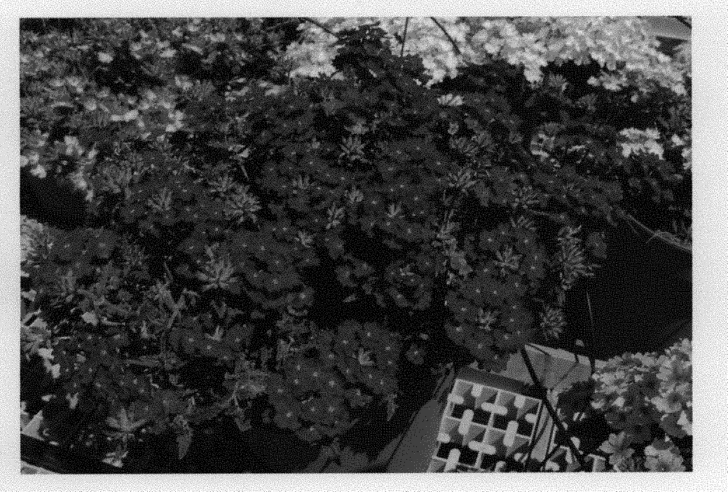

Genus and species: Verbena x hybrida.

Variety denomination: ‘KLEVP16485’.

### BACKGROUND OF THE NEW PLANT

The present invention comprises a new and distinct variety of verbena, botanically known as Verbena x hybrida, and hereinafter referred to by the variety name ‘KLEVP16485’. ‘KLEVP16485’ originated from a cross-pollination conducted in September 2010 in Cobbitty, New South Wales, Australia between the proprietary female verbena variety ‘VC10RB’ (unpatented), and the proprietary male verbena variety ‘VC11ABV’ (unpatented).

The seeds from the cross were sown and plants were grown for evaluation, where an individual plant designated ‘KLEVP16485’ was selected from the group of plants in Cobbitty, New South Wales, Australia in October 2011.

In November 2011, ‘KLEVP16485’ was first vegetatively propagated by stem cuttings in Cobbitty, NSW, Australia. ‘KLEVP16485’ was found to reproduce true to type in successive generations of asexual propagation via vegetative stem cuttings.

### SUMMARY

The following are the most outstanding and distinguishing characteristics of this new variety when grown under normal horticultural practices in Cobbitty, New South Wales, Australia.
1. Dark violet flowers with white eyes;
2. A semi-upright growth habit; and
3. Good tolerance to powdery mildew.

### DESCRIPTION OF THE PHOTOGRAPH

This new verbena plant is illustrated by the accompanying photograph which shows the plant's foliage and flowers.

The photograph is of a 15-week-old plant grown in a greenhouse in Stuttgart, Germany in April 2014. The colors shown are as true as can be reasonably obtained by conventional photographic procedures.

### DESCRIPTION OF THE NEW VARIETY

The following detailed descriptions set forth the distinctive characteristics of ‘KLEVP16485’. The data which define these characteristics were collected from asexual reproductions carried out in Cobbitty, New South Wales, Australia. Data was collected on three-month-old plants grown in a greenhouse in Cobbitty, New South Wales, Australia in June 2015. Color references are to The R.H.S. Colour Chart of The Royal Horticultural Society of London (R.H.S.), 5^{th} edition (2007).
Classification:
   Family.—Verbenaceae.
   Botanical.—Verbena x hybrida.
   Common.—Verbena.
   Designation.—‘KLEVP16485’.
Parentage:
   Female parent.—The proprietary female verbena variety ‘VC10RB’ (unpatented).
   Male parent.—The proprietary male verbena variety ‘VC11ABV’ (unpatented).
Plant:
   Growth and branching habit.—Semi-upright.
   Height (from top of soil).—32.0 cm.
   Width (horizontal plant diameter).—41.0 cm.
   Time to produce a finished flowering plant.—Approximately 8 weeks.
   Time to initiate and develop roots.—3 weeks.
   Root description.—Fine and fibrous roots, colored white, RHS 155B.
Stems:
   Number of branches per plant.—More than 16.
   Length.—20.0 cm.
   Diameter.—1.2 cm.
   Internode length.—2.1 cm.
   Color.—RHS 130D.
   Anthocyanin.—Absent.
   Texture and appearance.—Pubescent, round.
Leaves:
   Arrangement.—Opposite.
   Immature and mature leaf color.—Upper surface: RHS 141A. Lower surface: RHS 141C.
   Length.—2.5 cm.
   Width.—1.5 cm.
   Shape.—Lanceolate.
   Apex.—Acute.
   Base.—Obtuse.
   Margin.—Crenate.
   Texture.—Upper surface: Leathery. Lower surface: Rugose.
   Venation type.—Pinnate.
   Venation color (both upper and lower surfaces).—RHS 141D.
   Petioles.—Length: 0.2 cm. Diameter: 0.1 cm. Color: RHS 141D. Texture: Leathery.
Flower buds:
   Shape.—Tall, cylindrical.
   Length.—0.6 cm.
   Diameter.—0.15 cm.
   Color (at tight bud).—RHS 93B.
Inflorescence:
   Blooming habit (flowering season).—Continuously.
   Lastingness of individual blooms on the plant.—2 weeks.
   Fragrance.—Absent.
   Inflorescence type.—Umbel.
   Length of inflorescence.—5.0 cm.
   Diameter of inflorescence.—6.0 cm.
   Number of florets per inflorescence.—More than 15.
   Peduncle.—Color: RHS 141A. Length: 1.0 cm. Diameter: 0.1 cm.
Florets:
   Length.—2.0 cm.
   Diameter.—3.8 cm.
   Texture.—Glossy.
   Form.—Single.
   Length.—2.0 cm.
   Diameter.—3.8 cm.
   Color.—Immature: Upper surface: RHS N88A with white eyes RHS N999D. Lower surface: RHS 90B. Mature: Upper surface: RHS 93B with white eyes RHS N999D. Lower surface: RHS N88C.
   Corolla tube length.—1.1 cm.
   Corolla tube color.—RHS 56C.
   Petals.—Length of lobe: 1.9 cm. Diameter of lobe: 2.0 cm. Lobe shape: Round. Apex: Acute. Base: Obtuse. Margin: Entire. Texture (both upper and lower surfaces): Glossy, satiny.
   Pedicels.—Not visible.
Sepals:
   Shape.—Lanceolate.
   Apex.—Acute.
   Base.—Obtuse.
   Margin.—Entire.
   Length.—2.0 cm.
   Diameter.—0.5 cm.
   Color.—RHS 141C.
   Texture (both upper and lower surfaces).—Hairy, dull.
Reproductive organs:
   Stamens.—Quantity: 4. Appearance: Shiny. Color: RHS 139D. Filament length: 0.9 cm. Filament diameter: 0.1 cm. Anther: Shape: Elliptic. Color: RHS 141D. Length: 0.8 cm. Diameter: 0.2 cm. Pollen amount: Sparse.
   Pistils.—Number: 1. Length: 1.1 cm. Diameter: 0.1 cm. Stigma: Color: RHS 141D. Appearance: Ovate. Length: 0.1 cm. Diameter: 0.1 cm. Style: Color: RHS 141D. Length: 0.9 cm. Diameter: 0.1 cm. Appearance: Long.
Fruit and seed set: None observed.
Disease and pest/insect resistance: Good tolerance to powdery mildew.

### COMPARISON WITH PARENTAL LINES AND KNOWN VARIETY

‘KLEVP16485’ is distinguished from its parents as described in Table 1.

TABLE 1
Comparison with Parental Lines

| | | | |
|---|---|---|---|
| Characteristic | ‘KLEVP16485’ | Female parent | Male parent |
| | | ‘VC10RB’ | ‘VC11ABV’ |
| Flower color | Dark violet with | Solid purple | Purple with eyes |
| | white eyes | | |
| Earliness | Early | Medium | Early |

When ‘KLEVP16485’ is compared to the commercial verbena ‘Empress Flair Violet Blue’ (patent status unknown), differences between the varieties are described in Table 2.

TABLE 2
Comparison with Commercial Variety

| | | |
|---|---|---|
| Characteristic | ‘KLEVP16485’ | ‘Empress Flair Violet |
| | | Blue’ |
| Flower color | Dark violet with | Solid purple with dark |
| | white eyes | purple eyes |
| Tolerance to | Good tolerance | Less tolerance than |
| powdery mildew | | ‘KLEVP16485’ |

I claim:

1. A new and distinct variety of verbena plant designated ‘KLEVP16485’ as illustrated and described herein.
